(19) United States
(12) Patent Application Publication
(10) Pub. No.: US 2026/0334834 A1
(43) Pub. Date: Oct. 29, 2026

(54) NON-INVASIVE BLADDER MONITOR ASSEMBLY

(71) Applicant: DryRise LLC, Provo, UT (US)

(72) Inventors: Preston David Thomas, Mayfield, UT (US); Benjamin Carter Sannar, Provo, UT (US); Hannah Cardon, Provo, UT (US)

(73) Assignee: DryRise LLC, Provo, UT (US)

(21) Appl. No.: 19/653,453

(22) Filed: Apr. 21, 2026

Related U.S. Application Data
(60) Provisional application No. 63/793,112, filed on Apr. 23, 2025.

(51) Int. Cl. A61B 5/20; A61B 5/00; A61B 5/01; A61B 5/053; A61B 5/11

(52) U.S. Cl. CPC A61B 5/202; A61B 5/01; A61B 5/053; A61B 5/11; A61B 5/6804; A61B 5/7275; A61B 5/7455; A61B 5/746; A61B 2560/0468; A61B 2562/0219; A61B 2562/0271

(57) ABSTRACT

This disclosure relates to a bladder monitor assembly for home use in active users and a method of using that monitor. The bladder monitor assembly includes mounting bands and electrode backings to keep electrodes such as sensors in contact with the skin. The bladder monitor controls the bladder monitor assembly and transmits bioimpedance measurement values (such as electrical impedance values) obtained from the sensors to a computing device. The computing device generates, with a machine learning model executing the measurement values, a certainty value associated with a likelihood that the user will have a urinary accident (leakage event). In response to the certainty value satisfying a risk threshold value, the computing system generates a control signal, which is transmitted to the bladder monitor assembly. In response to the control signal, the bladder monitor assembly generates an alert stimulus that prompts the user to take action to avoid a leakage event.

### RELATED APPLICATION

This application claims priority to, and the benefit of, U.S. Provisional Patent Application No. 63/793,112, filed Apr. 23, 2025, and entitled “Non-Invasive Bladder Monitor,” which is hereby incorporated by reference in its entirety.

### BACKGROUND

Urinary incontinence is a common health condition that can significantly impact quality of life. Urinary incontinence can be managed with a monitor that alerts the user or a caretaker in advance of a leakage event occurring. However, current methods are invasive, requiring insertion of a device, like a catheter, into the user, or cumbersome, requiring that the patient remain completely still. Additionally, some monitors require complex steps to use the monitor, such as applying gel for an ultrasound monitor, which requires the aid of a professional healthcare provider or technical knowledge. Further, current monitors do not allow adjustment to notification settings. A mobile user may desire to adjust the alert stimulus of the monitor depending on whether the user is awake and active or asleep. For example, a user who wishes to be notified in advance of a leakage event while sleeping may want to receive a tactile stimulus, as an alert stimulus, that gradually increases in intensity to gently wake the user while an awake user may prefer an alert stimulus in the form of a phone notification to the mobile device. Additionally, existing approaches attempt to estimate absolute bladder volume, which requires complex modeling, extensive computational resources, and calibration procedures that are impractical for a portable, wearable device in a home setting.

### BRIEF DESCRIPTION OF THE DRAWINGS

In order to describe the manner in which the above recited and other advantages and features of the present disclosure can be obtained, a more particular description of the present disclosure briefly described above will be rendered by reference to specific embodiments thereof, which are illustrated in the appended drawings. Understanding that these drawings depict only typical embodiments of the present disclosure and are not therefore to be considered to be limiting of its scope, the present disclosure will be described and explained with additional specificity and detail through the use of the accompanying drawings, which are listed below.

FIG. 1 is a block diagram of an exemplary bladder monitor assembly according to some embodiments.

FIG. 2 is a conceptual diagram of a user wearing a bladder monitor while being active according to some embodiments.

FIG. 3 is a flowchart of an exemplary method for monitoring bladder conditions on a bladder monitor according to some embodiments.

FIG. 4 is a flowchart of an exemplary method for training or retraining a machine learning model on a cloud-based server according to some embodiments.

FIG. 5 is a block diagram of an exemplary bladder monitor assembly according to some embodiments wherein the bladder monitor communicates with a mobile device directly.

FIG. 6 is a conceptual diagram of the placement of electrodes on the user for measurement of bioimpedance according to some embodiments.

FIG. 7 is a conceptual diagram of the placement of electrodes on the user for measurement of bioimpedance according to some embodiments.

FIG. 8 is a conceptual cross section of a mounting band showing the mechanism for holding electrodes in place using electrode backings.

FIG. 9 is a flowchart of an exemplary method for monitoring bladder conditions on a bladder monitor according to some embodiments, wherein the bladder monitor retrains automatically.

### DETAILED DESCRIPTION

Aspects of the disclosure are provided in the following description and related drawings directed to various examples provided for illustration purposes. The words “exemplary” and/or “example” are used herein to mean “serving as an example, instance, or illustration.” Any aspect described herein as “exemplary” and/or “example” is not necessarily to be construed as preferred or advantageous over other aspects. Likewise, the term “aspect of the disclosure” does not require that all aspects of the disclosure include the discussed feature, advantage or mode or operation. The words ‘fed’ and ‘feed,’ when used in conjunction with machine learning and data or information, mean putting the data in the machine learning model for training or refinement of the machine learning model.

Certain bladder monitor systems employ invasive catheterization techniques. Catheterization requires insertion of a tube into the bladder. These invasive approaches are generally suitable only for immobile patients, confined to beds or wheelchairs. Conventional systems are not designed for active users. These restrictions limit the independence of individuals requiring bladder monitoring. Users cannot perform routine tasks without assistance. Additionally, invasive catheterization introduces infection risk to patients such as urinary tract infections. Alternative non-invasive approaches include ultrasound devices and near-infrared spectroscopy. Conventional ultrasound devices require trained technicians for operation. These devices require periodic manual measurements by trained personnel. Continuous monitoring cannot be achieved with current ultrasound technology. Near-infrared spectroscopy employs light absorption for bladder sensing. Adipose tissue attenuates near-infrared signals. Volume estimation has not been realized in clinical applications. Bioimpedance analysis measures electrical impedances of biological tissues and can be used to monitor bladder levels. Existing hardware systems use electrode methods not optimized for bladder monitoring at home because their methods focus on measuring real-time bladder volume—a process requiring an immobile patient and complex calibration—rather than prediction of when future leakage accidents occur.

The current bladder monitoring art does not solve these problems. Some systems use ultrasound transducers to measure bladder volume. Ultrasound transducers require rigid piezoelectric elements, acoustic coupling gel or pads, and precise positioning to maintain acoustic contact with the skin, making them less suitable for integration into a flexible, comfortable wearable band that can be worn continuously during daily activities and sleep. Additionally, although some systems can provide alerts when a threshold volume is reached, they do not incorporate sleep-state monitoring or machine learning-based prediction of optimal wake times to minimize sleep disruption while preventing incontinence events.

Although research has been conducted implementing wearable bioimpedance devices for measuring bladder volume, these devices have failed to address the issue of patients who move during measurement, sometimes detecting larger signals during movement than the difference between a full and an empty bladder and usually instructing the patient to hold entirely still, which may limit the ability to breathe. Thus, other research resulted in embedding the electrodes, which embedding is invasive and not suitable for home use.

Aspects of the present disclosure address these deficiencies in current blader monitor systems by employing a bladder monitor system using mounting bands to keep the electrodes in contact with the skin, removing the need for adhesive attachment or embedding the device. For example, a mounting band may be configured to be attached to a body of a user. A first electrode backing and a second electrode backing may be attached to the mounting band. A processor may also be attached to the mounting band. At least one bioimpedance sensor may be coupled to the processor. The bioimpedance sensor may be attached to the first electrode backing and configured to be held in contact with a skin surface and at a first location of on the body by force from the first electrode backing. In embodiments, the bioimpedance sensor measures an electrical impedance value in response to a current emitted by a drive electrode held in contact with the skin surface at a second location of by the second electrode backing.

In some embodiments, a communication interface is coupled to the processor and configured to transmit, to a computing system, data including the electrical impedance value useable by the computing system as an input to a machine learning model that generates a certainty value of a leakage event. The communication interface may also receive, from the computing system, a control signal in response to the certainty value satisfying a risk threshold value. The processor may be configured to cause, in response to the control signal, an alert stimulus to be generated that prompts the user to take action to avoid a leakage event. In varying the embodiments, the computing system is integrated with the bladder model assembly, a mobile device of the user or a cloud-based server with which the communication interface is communicatively coupled.

In some embodiments, the bladder monitor system may be operated by first measuring bioimpedance of the user, preferably the abdominal area. Then the measurements are sent to a computing system which uses the data as input for a machine learning model that generates a risk threshold value, or probability of an accident happening in the near future. If the certainty value reaches a threshold, the user may be prompted to take action to avoid a leakage event (an incontinence accident). In some embodiments the user is prompted using an alert stimulus such as an audible alarm or haptic feedback.

In some embodiments, the computing system has components to process and non-transitorily store computer-readable instructions. In some embodiments, these instructions govern the operation of the bladder monitor system. In some embodiments, the computing system is instructed to receive measurements comprising the bioimpedance of the user, input those measurements into a machine learning model that determines a certainty value of a leakage event occurring. In some embodiments, the computing system is instructed to compare that certainty value to a risk threshold value, and, if it exceeds the threshold value, to generate and transmit a control signal that triggers one of a bladder monitor assembly and a mobile device to prompt, via an alert stimulus, the user to take action to avoid a leakage event.

This innovation fixes the problems that prior art did not, making bioimpedance monitoring of bladder conditions accessible to home users without medical staff such as hospice, home nurses, or other home medical care. This device is also useable by mobile users because of its ease to wear and it is computationally efficient because it may skip the unnecessary step of calculating bladder volumes. Additionally, by putting the electrodes in contact with the users' skin without attaching it via gel or the like it is easier to wear. Separating the training computing system and the bladder monitor may further reduce computational resource and power capacity needs for the bladder monitor Finally, electrode backings help the electrodes to remain in place for mobile users and motion detectors prevent the detrimental effects of motion artifacts.

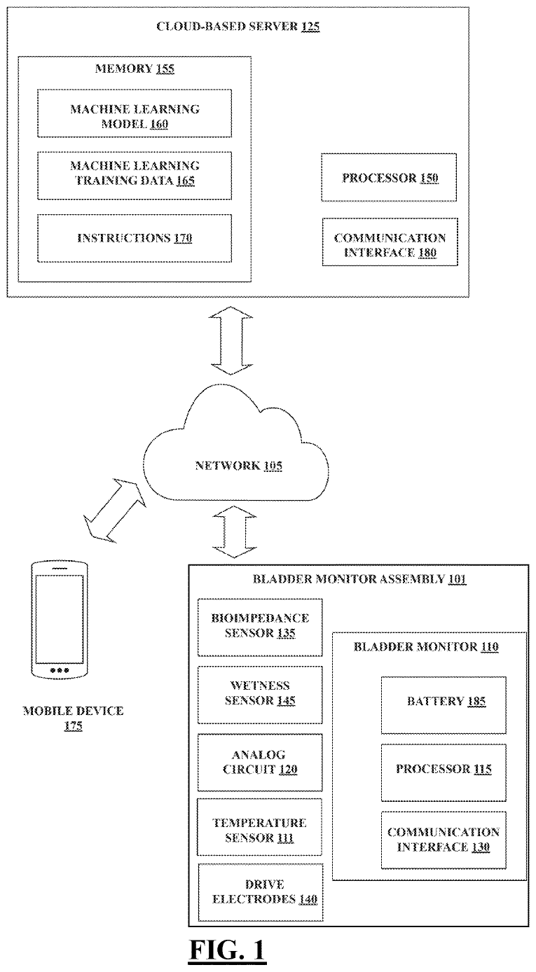

FIG. 1 illustrates a block diagram of an exemplary bladder monitor system 100, according to some embodiments. The bladder monitor system 100 includes a network 105, a cloud-based server 125, a bladder monitor assembly 101, and a mobile device 175. In some embodiments the bladder monitor assembly 101 may include a bladder monitor 110, an analog circuit 120, a bioimpedance sensor(s) 135, and a wetness sensor 145. The bladder monitor 110 may include a communications interface 130, and a processor 115. The network 105 may include wired and/or wireless communications according to one or more standards via one or more transport mediums. Communication over the network 105 may be in accordance with the various communication protocols, such as transmission control protocol and internet protocol (TCP/IP), user datagram protocol (UDP), and Institute of Electrical and Electronics Engineers (IEEE) communication protocols. The network 105 may further include wireless communications according to Bluetooth® specification sets, or another standard or proprietary wireless communication protocol. The network 105 may further include communications over a cellular network, including, for example, a global system for mobile (GSM) communications, code division multiple access (CDMA), and enhanced data for global evolution network (EDGE). The examples of the network 105 may include, but are not limited to, private or public local area network (LAN), wireless LAN (WLAN), metropolitan area network (MAN), and wide area network (WAN). The network 105 provides communication (e.g., connectivity) to the Internet (e.g., the cloud).

In some embodiments, the bladder monitor 110 may communicate with a mobile device 175 via a short-range wireless connection. In some embodiments, the mobile device 175 may further communicate with a cloud-based server 125 via a network 105. The network 105 may include wired and/or wireless communications according to one or more communication protocols, such as transmission control protocol and internet protocol (TCP/IP), user datagram protocol (UDP), or cellular communication protocols including global system for mobile communications (GSM), code division multiple access (CDMA), or long-term evolution (LTE). In some embodiments, the bladder monitor system 100 may operate independently of the network 105, with the mobile device 175 performing analysis and generating alerts using only data received via the short-range wireless connection. In some embodiments, both the mobile device 175 and the network 105 are in direct communication with the bladder monitor 110.

In some embodiments, the bladder monitor 110 includes a processor 115, a bladder monitor 110 and a communication interface 130. The bladder monitor 110 may communicate with the network 105 via the communication interface 130. Bluetooth®, Wi-Fi®, and other types of wireless interfaces may be examples of the communication interface 130.

The cloud-based server 125 includes memory 155, a processor 150, and a communication interface 180. HTTP/HTTPS and MQTT may be examples of the communication interface 180. A machine learning model 160, machine learning training data 165, and instructions 170 are examples of data and/or programs stored in the memory 155 and executable on the processor 150. In some embodiments, the machine learning model 160 may be a recurrent neural network (RNN). In some embodiments, the machine learning model 160 uses supervised learning to predict leakage events. In some embodiments, classification or regression is used for supervised learning in the machine learning model 160. In some embodiments, the model may be an AWS model in the cloud, an Android model or an iOS model stored in the codebase of the mobile device. In some embodiments, the training of the machine learning model 160 occurs in the cloud, on the bladder monitor, or on the mobile device. In some embodiments the machine learning model 160 may be a Long Short-Term Memory (LSTM) Model or Gated Recurrent Unit (GRU) architecture. In some embodiments, the machine learning model 160 is based on a classifier neural network architecture. These models deliver a certainty score based on an input. In some embodiments, the machine learning model operates as a classifier that receives a sequence of sensor measurements as input and outputs a certainty score. In some embodiments, the machine learning model 160 processes a sequence of sensor measurements such as bioimpedance, accelerometer, thermometer, clock, etc., and produces not only a certainty score for the present, but a projection of certainty scores into the future. In some embodiments batches of two hundred datapoints are fed into the machine learning model 160 to get a certainty value. In some embodiments the machine learning data consists of three thousand datapoints. In some embodiments, the measurements comprise around ten minutes of data. In some embodiments, the time period assessed for risk may be the next forty minutes allowing the user ample time to find somewhere to void (i.e. go to the bathroom). The machine learning model may analyze a series of past measurements to produce a certainty score that corresponds to the real-time likelihood that the user will have the need to void. In some embodiments the machine learning model 160 may do so without calculating a volume and simply report its prediction of a urination event occurring. In some embodiments, the machine learning model takes the percentage of measurement values predicting a leakage event and converts it into a certainty value from zero to one. In some embodiments the machine learning model 160 takes a large number of datapoints and output logits that are mathematically converted into a certainty value. Certainty value over or below a risk threshold value may be used to make binary True/False classifications. A processing device may include a processor. The processor 150 may be a central processing unit (CPU) within the cloud-based server 125 that may carry out instructions 170 by performing the basic arithmetical, logical, and/or input/output operations, according to several embodiments. The memory 155 may be a physical device used to store programs and/or data on a temporary and/or permanent basis for use in the cloud-based server 125, according to one embodiment. The cloud-based server 125 receives data from the bladder monitor 110 through the network 105 and may store the data in the memory 155. The machine learning model 160, when executed on the processor 150, determines a probability of the user having a leakage event within a set time period. If the probability of a leakage event occurring is above a threshold value, the cloud-based server 125 transmits a control signal to the bladder monitor 110 to alert the user. In some embodiments, the machine learning model 160 determines the certainty value based on one or more electrical impedance measurements taken by the bioimpedance sensor(s) 135. The electrical impedance measurements may be inputs to the machine learning model 160. Additionally, or alternatively, the machine learning model 160 may determine the certainty value based on one or more measurements from the wetness sensor 145. Accordingly, the moisture measurements from the wetness sensor 145 may be inputs to training of the machine learning model 160 or the operation of the machine learning model 160. In some embodiments, the time of day, the time between voiding events, the activity of the person, are used to train the machine learning model 160 or fed into a machine learning model 160 prompting it to calculate a certainty value. In some embodiments the machine learning model 160 can predict the next forty minutes of probabilities with the last ten minutes of data. In some embodiments, the data of the machine learning model 160 is cleaned by excluding data points based on simultaneous motion data from the accelerometer before training or predicting a certainty score. In some embodiments time is normalized by time of day or time since the last urination. In some embodiments the model hyperparameters are from approximately 64×1×20 GRU encoder to 64×1×80 GRU decoder. In some embodiments the model hyperparameters are 64×1 linear layer outputting a certainty series. In some embodiments, the data is standardized by converting all measurements to z-scores by dividing the difference between each value and the mean by the standard deviation. In some embodiments the hidden state size for training is sixty-four. In some embodiments there is one recurrent layer. In some embodiments there is one linear layer. In some embodiments, the training data 165 are scored by a sigmoid function and corrected using a binary cross entropy loss function to punish the machine learning model 160 confidently predicting incorrectly.

Volume is a relevant factor for leakage but is not the only one and it cannot be measured directly. In some embodiments, the computing system—which can be a laptop, desktop computer, mobile device, smartwatch, smart ring, glasses, the bladder monitor, or the cloud-based server—does not calculate volume because doing so is unnecessary and unlikely to improve the predictions of a leakage event. From a linear algebra lens, volume (which cannot be measured directly) and bioimpedance (which are measured by the bioimpedance sensor(s) 135) are linearly dependent. Because of this, measuring volume is feasible through linear transformations of impedance, but it does not provide any new information. Because there is no new information to be gained from volume, it may be unnecessary to calculate or measure it. Instead, the machine learning model 160 may incorporate linearly independent factors like time and other biometrics in series. The machine learning model 160 may learn the interrelated dynamics of these biometrics with respect to time and signal quality (i.e. by the motion detector) in order to sort through noise and output a series of predicted certainties for timesteps into the future.

In some embodiments, the machine learning training data 165 includes measurements from the bioimpedance sensor(s) 135 and the wetness sensor 145. In other embodiments, the machine learning training data 165 includes negative feedback from the user such as false or missed notifications. In other embodiments, the machine learning training data 165 includes positive feedback, such as allowing the user to sleep without a leakage event or properly detecting a leakage event. The bladder monitor 110 sends multiple impedance and wetness measurements to the cloud-based server 125 over a period of time. The cloud-based server 125 may store the measurements as machine learning training data 165. Accordingly, the first set of measurements sent from the bladder monitor 110 to the cloud-based server 125 may be used to train the machine learning model 160 and the second set of measurements sent from the bladder monitor 110 to the cloud-based server 125 may be used as inputs to the machine learning model 160 to determine the probability of a leakage event occurring. Additionally, or alternatively, the machine learning training data 165 may not include measurements from the current user of the bladder monitor assembly 101. For example, the machine learning training data 165 may be information uploaded by the user via the mobile device 175. In such embodiments, the first set of measurements received by the cloud-based server 125 may become inputs to the machine learning model 160 to determine the probability of a leakage event occurring. The machine learning model 160, when initially executed by the processor 150, is initialized with the machine learning training data 165.

In some embodiments, the processor 115 is coupled to the analog circuit 120, the bioimpedance sensor(s) 135, and the wetness sensor 145. In some embodiments, the bladder monitor assembly 101 includes a battery monitor, LEDs, lights or any other interface to monitor power levels of a battery 185 of the bladder monitor 110. The processor 115 may send one or more control signals to initialize the bioimpedance sensor(s) 135, the wetness sensor 145, and the analog circuit 120. For example, the processor 115 may send a control signal to configure the bioimpedance sensor(s) 135 with a defined time interval for taking measurements. Additionally, or alternatively, the processor 115 may send a control signal to configure the wetness sensor 145 with a time interval for taking measurements. The bioimpedance sensor(s) 135 may contact a skin surface of the user and measure an electrical impedance of a body (or portion of the body) of the user.

In some embodiments, the bioimpedance sensor(s) 135 is configured to take measurements at defined time intervals (e.g., every 3, 5, 8, or 10 minutes depending on application and user), and the bioimpedance sensor(s) 135 sends the measurement to the processor 115. The bladder monitor 110 sends the measurement to the network 105 via the communication interface 130, and the network 105 sends the measurement to the cloud-based server 125 via the communication interface 180. In some embodiments, the bladder monitor 110 sends the measurement to the network 105 when the impedance drops below a threshold value. In various embodiments, the communication interface 180 is configured to communicate using TCP/IP and/or HTTP/HTTPS and/or MQTT technologies. The machine learning model 160 may determine the probability of the user having leakage event based on the received electrical impedance measurement. In some embodiments, the cloud-based server 125 may transmit an indication of a leakage event to the network 105 when the cloud-based server 125 determines the user is close to having a leakage event (e.g., within 10 minutes of having a leakage event), and the cloud based server 125 transmits the indication to the bladder monitor 110. In some embodiments, another computing device, such as a mobile device 175 may determine the user is close to having a leakage event and transmit an indication or indicate the user directly. The processor 115 may then generate a control signal to transmit to the analog circuit when the bladder monitor 110 receives the indication over the network 105 from the cloud-based server 125.

In some embodiments, a sliding window of bioimpedance measurements preceding a leakage event detected by the wetness sensor 145 is labeled as a positive training example, and windows not associated with a leakage event within a defined prediction horizon are labeled as negative training examples. In some embodiments, the prediction horizon corresponds to the defined time interval between measurements multiplied by a configurable number of future time steps.

In some embodiments, the drive electrodes 140 may be made of a metallic material such as stainless steel, gold, or titanium. In other embodiments, the drive electrodes 140 may be made of a conductive polymer. In other embodiments, the drive electrodes 140 may be made of carbon rubber, conductive textiles, or microelectrodes. In other embodiments the drive electrodes 140 are a mix of silver nanowires or thin film metals and embedded in a flexible substrate like polyimide, or silicone. In some embodiments, the drive electrodes 140 produce a positive alternating current, a negative alternating current, or an alternating current that oscillates between being positive and negative. In some embodiments, the polarity or phase of the drive electrodes 140 are opposite to one another. In some embodiments, the frequency of the alternating current is from a few kHz to several MHz. In some embodiments the current is a low amplitude in the microamp (μA) to the 2 mA range when measuring.

In some embodiments, the bioimpedance sensor(s) 135 may be made of a metallic material such as stainless steel, gold, or titanium. In other embodiments, the bioimpedance sensor(s) 135 may be made of a conductive polymer. In other embodiments, the bioimpedance sensor(s) 135 may be made of carbon rubber, conductive textiles, or microelectrodes. In other embodiments the bioimpedance sensor(s) 135 are a mix of silver nanowires or thin film metals and embedded in a flexible substrate like polyimide, or silicone. In some embodiments, the bioimpedance sensor(s) 135 detect voltage drops or gains in or near the mV range. In some embodiments, the bioimpedance sensor(s) 135 detect voltage drops or gains in the opposite phase or polarity from one another.

The bioimpedance sensor(s) 135 and the drive electrodes 140 may be placed in various configurations. In some embodiments, the outer electrodes are the drive electrodes 140. In other embodiments, the inner electrodes drive current. In some embodiments the drive electrodes 140 are placed in the same parasagittal plane as each other. In some embodiments, the drive electrodes 140 are placed in the same parasagittal plane as a bioimpedance sensor(s) 135. In some embodiments, the inputs to the machine learning model 160 may also include frequency of the current, resistance, reactance, magnitude of reactance, phase degrees, temperature, standard deviation in the ohms, quality of the measurement, time, motion in one or more directions, or a urination event. In some embodiments, all measurements and labels, are delivered to the cloud. Bulk data from all users may be used to refine a base model and condition specific-variants. A user's own measurements may be used to further tune their personal algorithm. Thus in some embodiments, the machine learning model 160 is a personal algorithm adapted from a base model refined by bulk data from all users. In some embodiments, updates may be pushed to the base model periodically or subject to terms, and user data may be used to further refine the user's experience. Frequency of updates, or a passage of predetermined time, may be determined by the user using user settings in the mobile app. In some embodiments, time series windowing, startup time, frequency-domain decomposition, or extraction of resistance or reactance components may be used to balance local and global patterns.

In some embodiments, the wetness sensor 145 is the drive electrodes 140. In some embodiments, the wetness sensor 145 is a separate capacitive, conductive, or resistive sensor, such as a leaf electrode, a RECT electrode, a RFID tag, electrodes, or textile electrodes comprising a conductive yarn such as graphite, silver, or any other conductive material.

In some embodiments, the temperature sensor 111 is a thermocouple, a resistance temperature detector, a thermistor, or a semiconductor-based ICs. In some embodiments the temperature sensor 111 uses the electrodes of the wetness sensor 145 or bioimpedance sensor(s) 135 to detect changes in resistance. In other embodiments, the temperature sensor 111 is a separate component. In some embodiments, the temperature sensor 111 measures body temperature. In other embodiments, the temperature sensor 111 measures room temperature.

In some embodiments, the analog circuit may use the drive electrodes. In some embodiments, the analog circuit 120 uses an alternative electrical connection to wake or alert the user. In some embodiments, the analog circuit 120 supplies a constant current in the 1-6 mA range when alerting or awaking the user. In some embodiments the analog circuit 120 is continuous. In other embodiments, the current is a series of spaced pulses in the 1-6 mA range. In some embodiments, amperage may be lower for women than men. In some embodiments, voltages and currents of the analog circuit 120, bioimpedance sensor(s) 135, and drive electrodes 140 can be configured by user.

In some embodiments, the bladder monitor assembly 101 may include a motion detector such as an accelerometer, a gyroscope, magnetometer, IMUs, a ball tilt switch, a MEMS microphone. The motion detector may generate motion values indicating the motion of the user or the mounting band. The motion values may be used to filter out the worst data points by, for example, a logical algorithm. This may further be used for applying training labels to the machine learning data 165 pertaining to measurement quality or type. The machine learning model 160 may be trained on accelerometer data allowing it to learn to adjust or deemphasize motion artifacts. In some embodiments, the inputs are cleaned before training by, for example, normalization or adding safety margins, filtering datapoints, or weighing moving datapoints less. In some embodiments, data may be labeled by the wetness sensor 145 or by the user's manual feedback to the mobile device or the bladder monitor. Manual feedback could include clicking a button or typing something into the app.

In some embodiments, the bladder monitor assembly 101 may be attached using a mounting band 205. In some embodiments, the mounting band 205 may include a cloth including cotton, polymer, rubber, leather or any other fabric. In some embodiments, the mounting band 205 may be secured to the user using a button, buckle, Velcro, elastics, magnets, hooks and loops, or the like. In other embodiments, the mounting band 205 may be an elastic or flexible material configured to stretch over the user and held in place by elasticity. In many embodiments, the tightness of the mounting band 205 may be increased by shortening a strap, attaching to different through holes. In some embodiments, consistent electrode placement may be achieved by aligning a through hole, window, marking, or other part of the mounting band 205 to a natural or artificial mark on a body of the user, such as a belly button, freckle, mole, other bodily feature, piercing, tattoo, or the like. In some embodiments, the mounting band 205 is a belt or band that fits around the circumference of a user's waist. In some embodiments, the mounting band may further comprise a mounting garment 605 such as briefs, panties, underwear, or shorts. In some embodiments the mounting band 205 may be a belt, EKG strap, harness, band, obi, corset, clothing, jumper, or other tight fitting or adjustable wearable article.

In some embodiments, the analog circuit 120 is attached to a mounting band 205 and is in physical contact with the user, e.g., with skin of the user. The processor 115 can send a control signal to the analog circuit 120 to turn the analog circuit 120 on and off. The analog circuit 120 can generate an alert stimulus to prompt the user to take action to avoid a leakage event (i.e. go to the bathroom). An alert stimulus may be a haptic feedback, a tactile stimulus such a current, an audio alert signal, or other alert stimulus capable of notifying and/or waking up the user or prompting the user, because the alert stimulus is detectable, to take action to avoid a leakage event. In some embodiments, the alert stimulus may be achieved through a vibrator or through the user's phone's vibrator, screen or ringer. In some embodiments, such as those dealing with nocturnal enuresis, electrical stimulation for users may provide stronger alert stimulus. In some embodiments, the alert stimulus is a vibration, an audible alarm, or an electric pulse. In some embodiments, the analog circuit 120 generates the alert stimulus for a defined time period. Additionally, or alternatively, the analog circuit 120 may generate the alert stimulus until the analog circuit 120 receives a control signal from the processor 115, which turns the analog circuit 120 off. For example, the user may reset the bladder monitor assembly 101 once the user goes to the bathroom, in which case the processor 115 may send a control signal to turn off the analog circuit 120. Turning off the analog circuit 120 may at least put the analog circuit 120 in a low-power mode waiting for a control signal to be turned back on again. Additionally, or alternatively, the processor 115 may send a control signal to turn off the analog circuit 120 if the user does not reset the bladder monitor 110 within a set time period. For example, the processor 115 may turn off the analog circuit 120 if the user is unresponsive to the alert stimulus (e.g., the user does not wake up to go to the bathroom). In some embodiments, the alert stimulus has variable intensity. For example, the analog circuit 120 may generate an electrical pulse that increases in intensity over a set period of time to encourage the user to take action to avoid a leakage event.

In some embodiments, the wetness sensor 145 is in physical contact with the user (e.g., skin of the user) and continuously measures moisture values of the user. The wetness sensor 145 may transmit a signal to the processor 115 when the moisture values exceeds a threshold value. For example, the wetness sensor 145 may alert the processor 115 when the measured moisture values indicates the user is close to having a leakage event, e.g., by measuring sufficient urinary leakage. In some embodiments these levels are measured by a lowering of resistance between two wires due to the presence of water. The bladder monitor 110 may transmit, over the network 105, the moisture measurement to the cloud-based server 125 via the communication interface 130 based on the signal sent to the processor 115. In some embodiments, the cloud-based server 125 stores and uses the moisture measurement as machine learning training data 165. In some embodiments, the machine learning training data 165 from the user may be used to update or fine tune a pretrained model. In some embodiments, the bladder monitor 110 sends multiple measurements from the wetness sensor 145 to the cloud-based server 125 over a set period of time to train the machine learning model 160. In some embodiments, the machine learning model is retrained when the moisture values measured by the wetness sensor 145 exceed a moisture value threshold. In some embodiments, the moisture value threshold is configurable. In some embodiments the moisture value threshold is above the normal moisture point generated by a normal amount of sweat. In some embodiments the user manually indicates via the mobile device 175 or the bladder monitor assembly 101, that a leakage event occurred. Additionally, or alternatively, the cloud-based server 125 may use the moisture measurements as inputs to the machine learning model 160 to determine a probability of a leakage event occurring. Additionally, in some embodiments, the moisture measurements may be used to label a leakage event if it reaches a moisture value threshold. In some embodiments, the leakage event may trigger retraining of the machine learning model.

In some embodiments, the user can configure and control the bladder monitor 110 using the mobile device 175. The mobile device 175 can transmit and receive data over the network 105 via a communication interface, e.g., using Bluetooth®, Wi-Fi®, LTE, or 5G technologies. The network 105 (e.g., an access point device of the network) may receive the signal from the mobile device 175 and transmit a signal to the bladder monitor 110 indicating the request from mobile device 175. The user may configure a power setting, specify a type of alert stimulus or reset the device. The mobile device 175 may send a signal to the network 105 indicating the request. The network 105 may receive the signal and transmit a signal to communication interface 130 of the bladder monitor 110 indicating the request from the mobile device 175. The processor 115 may generate one or more control signals, based on the request, and transmit the control signals to the bioimpedance sensor(s) 135, the wetness sensor 145, and the analog circuit 120. For example, the mobile device 175 may request the bladder monitor assembly 101 operate in a low power mode. The bladder monitor 110 may receive the request and the processor 115 may generate a control signal configuring the bioimpedance sensor(s) 135 to take measurements at less frequent time intervals and disabling the wetness sensor 145. In another example, the mobile device 175 (e.g., via user selection in an application running on the mobile device 175) may transmit a request to the bladder monitor 110 that the user be alerted by a notification on the mobile device 175 rather than by a vibration or electric shock via the analog circuit 120. Accordingly, the bladder monitor 110 may receive the request and the processor 115 may generate a control signal disabling the analog circuit 120 based on this user selection.

In some embodiments, the bladder monitor 110 is communicatively coupled with some or all of the components of the bladder monitor assembly 101, such as the bioimpedance sensor(s) 135, the wetness sensor 145, or the drive electrode(s) 140.

In some embodiments, the processor 115 may send and receive data (such as a battery indicator or power level) from the battery 185 of the bladder monitor 110. A battery monitor or interface can monitor the power levels of the bladder monitor 110 and send data indicating a power level of the bladder monitor 110 to the processor 115. In some embodiments, the processor 115 generates one or more control signals in response to receiving the battery indicator from the battery monitor or interface. For example, the processor 115 may send a control signal to the bioimpedance sensor(s) 135 to reduce the frequency of measurements if the power is low. Additionally, or alternatively, the bladder monitor 110 may send data from the battery indicator or power level to the network 105, which may cause the network 105 to send a notification to the mobile device 175 notifying the user that power is low. In other embodiments, the machine learning model 160 is located on the mobile device 175, to avoid the need for constant connection to the internet.

In some embodiments, the bladder monitor 110 sends unmodified data (e.g., measurement data) to the cloud-based server 125. In other embodiments, the processor 115 modifies data received from the bioimpedance sensor(s) 135 and the wetness sensor 145 before the bladder monitor 110 transmits the measurements to the network 105. Additionally, or alternatively, the bladder monitor 110 may cache the data locally. For example, if the communication interface 130 is disabled, the bladder monitor 110 may cache data until communication with the network 105 is reestablished and the data can be transmitted.

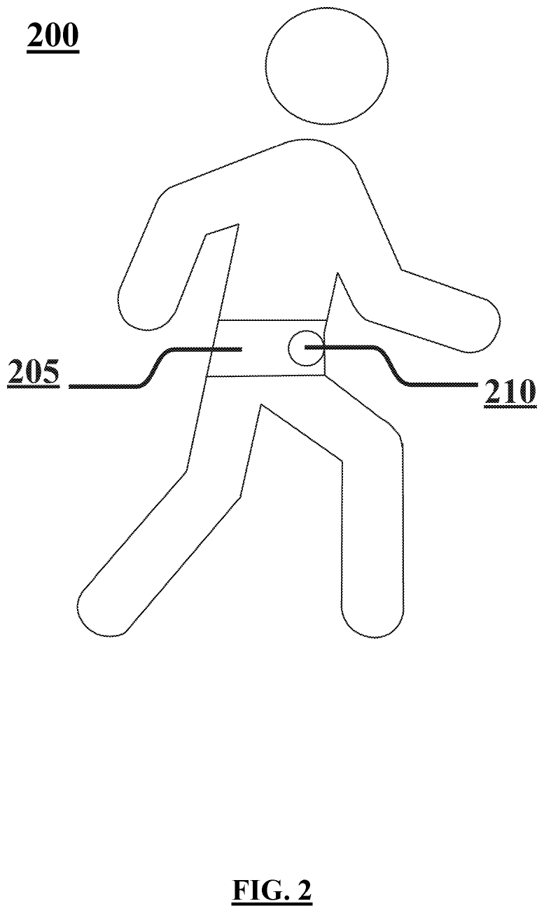

FIG. 2 illustrates a diagram 200 of a user wearing a bladder monitor while being active, according to some embodiments, including a mounting band 205 and a bladder monitor 210. In some embodiments, the bladder monitor 210 is the bladder monitor 110 shown in FIG. 1. For example, the processor 115, bioimpedance sensor(s) 135, wetness sensor 145, and the analog circuit 120 may be attached to or integrated within the mounting band 205. The mounting band 205 may be attached to the user. In some embodiments, the mounting band 205 may be a wearable garment that surrounds the pelvic area of the user. Additionally, or alternatively, the mounting band 205 may be a sticky pad that adheres to the abdominal area of the user and thus attaches the electrode to the skin in order to maintain contact. The mounting band 205 may provide support to the bladder monitor 210. For example, the mounting band 205 may prevent the bladder monitor 210 from shifting or moving away from the pelvic area as the user moves around, which may result in more accurate sensor measurements. The mounting band 205 may keep the bladder monitor 210 in place even when the user engages in highly active movements such as walking or running. The mounting band 205 may also provide a greater surface area of contact for the electrodes of the bladder monitor 110 and the skin surface of the user, which may reduce sensor noise and increase measurement accuracy. The mounting band 205 may also provide the user with easy access to the bladder monitor 210 by being easy to put on and remove. The mounting band 205 allows the bioimpedance sensor(s) 135 and wetness sensor 145 to be in contact with a skin surface of the user.

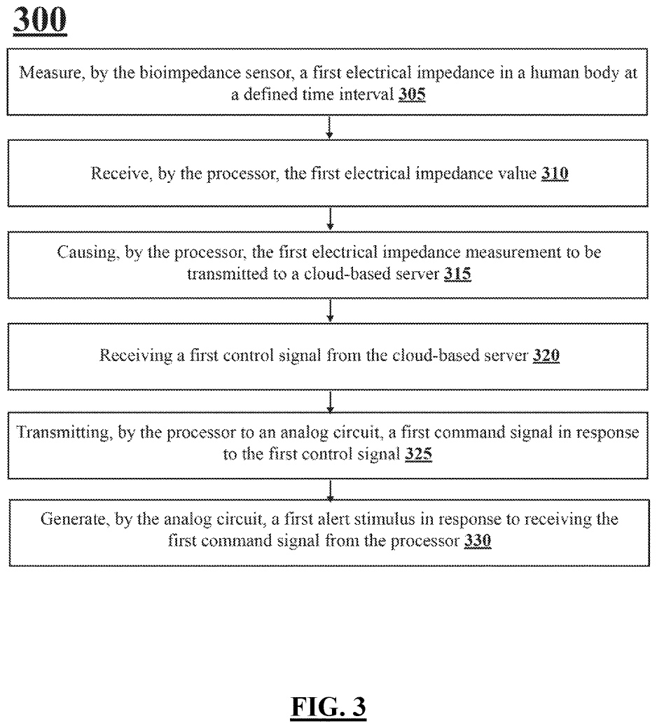

FIG. 3 illustrates a flowchart of an example method 300 for monitoring bladder conditions at a bladder monitor according to some embodiments. The method may be performed by a bladder monitor (such as the bladder monitor 110 shown in FIG. 1 and/or the bladder monitor 210 shown in FIG. 2). The method 300 may be performed by processing logic that may include hardware (e.g., processing device, circuitry, dedicated logic, programmable logic, microcode, hardware of a device, integrated circuit, etc.), software (e.g., instructions run or executed on a processing device), or a combination thereof. In an illustrative example, the method 300 is performed by the bladder monitor 110 of FIG. 1 and the processing logic can be most associated with the processor 115.

Although shown in a particular sequence or order, unless otherwise specified, the order of the operations may be modified. Thus, the illustrated implementations should be understood only as examples, and the illustrated operations may be performed in a different order, while some operations may be performed in parallel. Additionally, one or more operations may be omitted in some implementations. Thus, not all illustrated operations are required in every implementation, and other process flows are possible.

At operation 305, the processing logic measures, using the bioimpedance sensor(s) 135, a first electrical impedance in a human body at a defined timer interval.

At operation 310, the processing logic receives the first electrical impedance value.

At operation 315, the processing logic causes the first electrical impedance measurement to be transmitted to the cloud-based server 125. The first electrical impedance value causes the cloud-based server 125 to calculate, using a trained machine learning model 160, a certainty value based on the first electrical impedance value.

In some embodiments, the machine learning model 160 is trained prior to the bladder monitor 110 transmitting the first electrical impedance measurement. The bioimpedance sensor(s) 135 can measure one or more electrical impedance values in the human body and the wetness sensor 145 can measure one or more moisture levels in the human body. The bioimpedance sensor(s) 135 and the wetness sensor 145 can transmit the measurements to the processor 115. In some embodiments, the processing logic causes the bladder monitor 110 to transmit, via the communication interface 130, the measurements to the cloud-based server 125 to be fed into the machine learning model 160. In some embodiments, the cloud-based server 125 stores the measurements as machine learning training data 165.

At operation 320, the processing logic receives a first control signal from cloud-based server 125 based at least in part on the certainty value exceeding the threshold value. In embodiments, the machine learning model 160 calculates the certainty value by taking the first electrical impedance measurement as input. The processor 150 may compare the output of the machine learning model 160 to the risk threshold value. If the certainty value exceeds the risk threshold value, the cloud-based server 125 may generate a control signal to transmit to the bladder monitor 110 to alert the user of a leakage event. In some embodiments the threshold value may be determined by the user after their own trial and error. In other embodiments the risk threshold value may be determined by the user who consults with their healthcare provider. In some embodiments, the threshold value may be set dynamically by the user, causing the threshold value to be replaced by the user defined threshold value during operation. In some embodiments, the risk threshold value is preset by the manufacturer.

At operation 325, the processing logic transmits a first command signal to the analog circuit 120 in response to receiving the first control signal. The first control signal may turn on the analog circuit 120.

At operation 330, the processing logic (e.g., the analog circuit 120) generates alert stimulus in response to receiving the first command signal from processor 115. For example, the analog circuit 120 may generate a vibration or a shock upon receiving the first control signal.

In some embodiments, the processing logic reconfigures analog circuit 120 based on a request from the user. For example, the bladder monitor 110 may receive a second control signal from the cloud-based server 125 in response to the cloud-based server 125 receiving a request from the mobile device 175 to reconfigure the analog circuit 120. In some embodiments, the user wants to change the alert stimulus from a vibration to an electric shock. In an example, the user may want to disable the analog circuit 120 entirely in favor of a receiving a notification on a mobile device 175. The processing logic may transmit to the analog circuit 120, a reconfiguration command in response to receiving the second control signal. In some embodiments, the analog circuit 120 reconfigures, in response to the reconfiguration command, alert stimulus circuitry of analog circuit 120 to generate second alert stimulus in lieu of the first alert stimulus. For example, the bladder monitor 110 may be configured to gently awaken the user when the user is sleeping. As such, the analog circuit 120 may initially be configured to generate the first alert stimulus as a gradually increasing vibration. When the user is awake, the user may reconfigure the bladder monitor 110 to provide an electric shock that can be felt while the user is busy moving around. As such, the reconfiguration command may configure the analog circuit 120 to generate an electric shock as the second alert stimulus. Accordingly, the first alert stimulus and the second alert stimulus may have different tactile sensations.

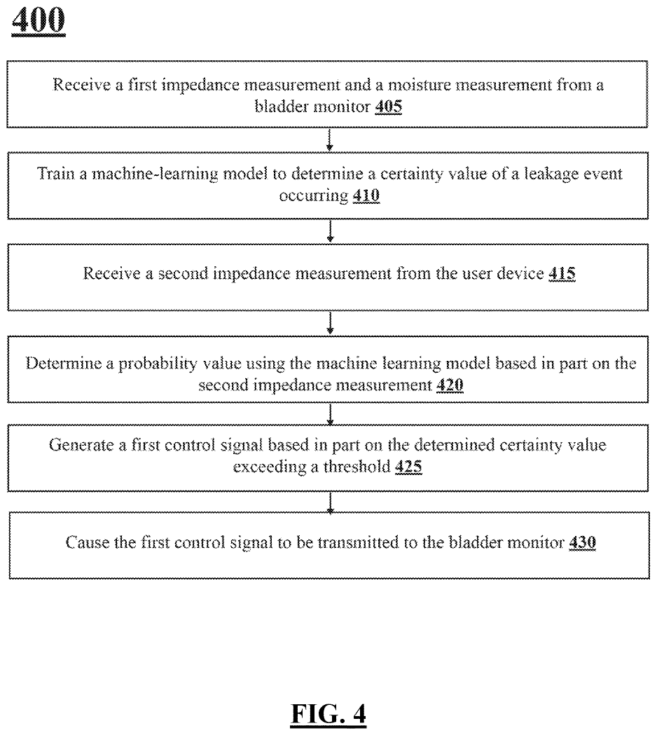

FIG. 4 illustrates a flowchart of an example method 400 for monitoring bladder conditions, which is executable by a processor of a cloud-based server according to some embodiments. The method 400 may be performed by processing logic that may include hardware (e.g., processing device, circuitry, dedicated logic, programmable logic, microcode, hardware of a device, integrated circuit, etc.), software (e.g., instructions run or executed on a processing device), or a combination thereof. In an illustrative example, the method 400 is performed by a cloud-based server (such as the cloud-based server 125 shown in FIG. 1).

Although shown in a particular sequence or order, unless otherwise specified, the order of the operations may be modified. Thus, the illustrated implementations should be understood only as examples, and the illustrated operations may be performed in a different order, while some operations may be performed in parallel. Additionally, one or more operations may be omitted in some implementations. Thus, not all illustrated operations are required in every implementation, and other process flows are possible.

At operation 405, the processing logic receives from the bladder monitor 110, a first impedance measurement and a moisture measurement. The cloud-based server 125 receives the measurements from the bladder monitor 110 via the communication interface 130 and the network 105.

At operation 410, the processing logic trains, based on the first impedance measurement and the moisture measurement, the machine learning model 160 to determine the certainty value of a leakage event occurring.

At operation 415, the processing logic receives from the bladder monitor 110, a second impedance measurement of the user taken at a defined time interval.

At operation 420, the processing logic determines, using the machine learning model 160, a certainty value of a leakage event occurring based at least in part on the second impedance measurement. For example, the processing logic may input the second impedance measurement into the machine learning model 160 and the machine learning model 160 may output a probability of the user having a leakage event within a set time period.

At operation 425, the processing logic generates a first control signal based in part on the determined certainty value exceeding a threshold. In some embodiments, the processing logic has a defined threshold value for alerting the user. If the certainty value is greater than the threshold value, the processing logic generates a control signal indicating to alert the user. If the certainty value is less than or equal to the threshold value, the processing logic either does not send a corresponding control signal or sends a control signal communicating to the bladder monitor 110 not to alert the user.

At operation 430, the processing logic causes the first control signal to be transmitted to the bladder monitor 110. The first control signal may in turn cause the bladder monitor assembly 101 to generate alert stimulus at the bladder monitor assembly 101.

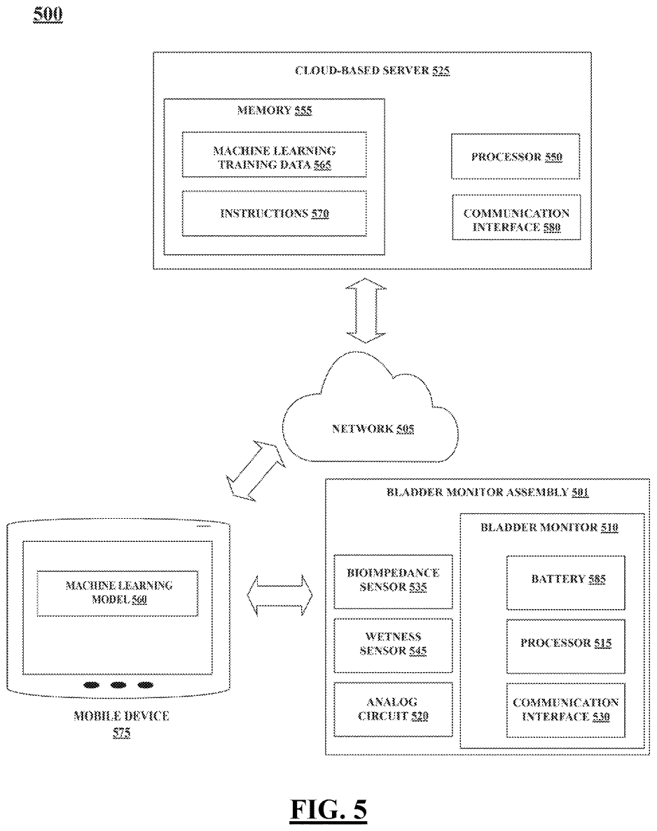

FIG. 5 shows an exemplary embodiment of a bladder monitor system 500, wherein the bladder monitor 510 communicates directly with the mobile device 575 using the communication interface 530. The mobile device 575 in turn connects with the cloud-based server 525 through the network 505. The bladder monitor assembly 501 may comprise a bioimpedance sensor 535, a battery 585, a wetness sensor 545, a bladder monitor 510, and a communication interface 530. The bladder monitor 510 may include a processor 515, and a communication interface 530. The cloud based server 525 may comprise memory 555, a processor 550, and a communication interface 580. The memory 555 may comprise a machine learning model 560, machine learning training data 565, and instructions 570. This embodiment can be more robust than the embodiment shown in FIG. 1 in situations where connecting to the cloud based server 525 by internet through cellular data, or Wi-Fi, is difficult. Additionally connecting directly to the bladder monitor 510 simplifies connection for the user because they do not need to connect the bladder monitor 510 to the internet. The connection between the bladder monitor 510 and the mobile device 575 is instead made directly using methods such as Bluetooth, and the mobile device 575 is connected to the internet via Wi-Fi®, LTE, 5G, or Ethernet.

In other embodiments, the machine learning model 560 is located on the mobile device 575, to avoid the need for constant connection to the internet.

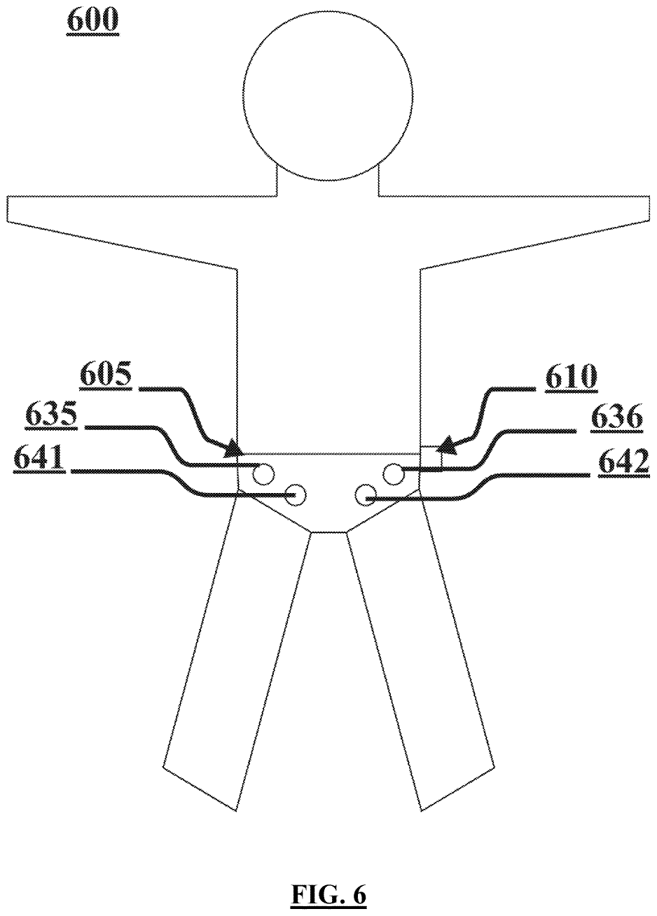

FIG. 6 shows an exemplary placement of sensors on a garment 605 in some embodiments. The bladder monitor assembly 600 may comprise a garment 605, a positive drive bioimpedance electrode 635, a negative drive bioimpedance electrode 636, a positive bioimpedance sensor 641, and a negative bioimpedance sensor 642. The positive drive bioimpedance electrode 635, the negative drive bioimpedance electrode 636, the positive bioimpedance sensor 641, and the negative bioimpedance sensor 642 may be placed on the abdomen of a user. Additionally, the bioimpedance sensors 641, 642 may be above, below, or at the same height as the drive bioimpedance electrodes 635, 636. Conducting wires in the garment 605 connect the sensors 641, 642 and electrodes 635, 636 to the bladder monitor 610. The bladder monitor 610, may further comprise a battery 185, a wetness sensor 145, a processor 115, an analog circuit 120, and a communication interface 130.

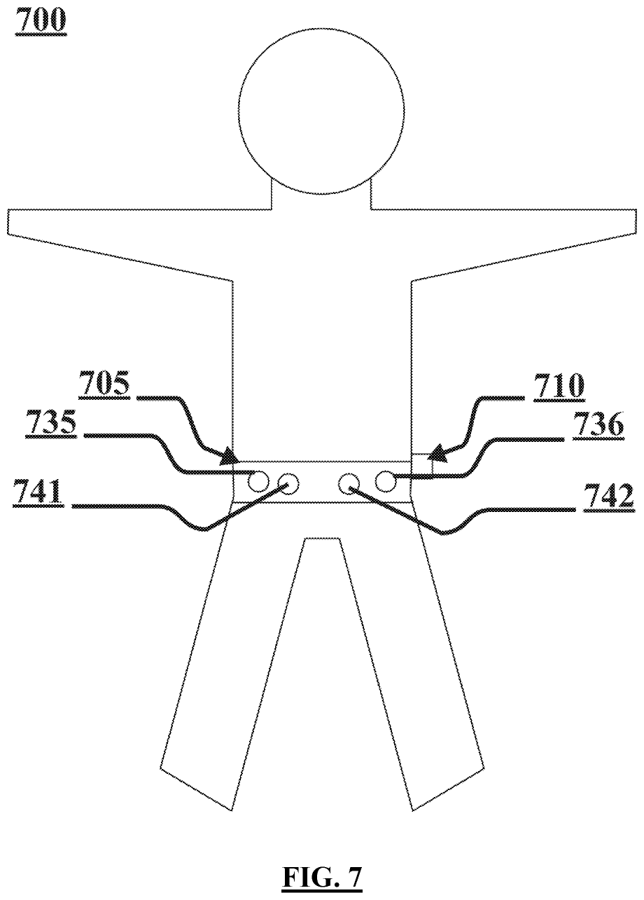

FIG. 7 shows an exemplary bladder monitor assembly 700 and shows the placement of sensors on a mounting band 705 in some embodiments. The bladder monitor assembly 700 may comprise the mounting band 705, a positive drive bioimpedance electrode 735, a negative drive bioimpedance electrode 736, a positive bioimpedance sensor 741, and a negative bioimpedance sensor 742. The positive drive bioimpedance electrode 735, the negative drive bioimpedance electrode 736, the positive bioimpedance sensor 741, and the negative bioimpedance sensor 742 may be placed on the abdomen of a user. Additionally, the bioimpedance sensors 741, 742 may be above, below, or at the same height as the drive bioimpedance electrodes 735, 736. Conducting wires in the mounting band 705 connect the sensors 741, 742 and electrodes 735, 736 to the bladder monitor 710. The bladder monitor 710, may further comprise a battery 185, a wetness sensor 145, a processor 115, an analog circuit 120, and a communication interface 130.

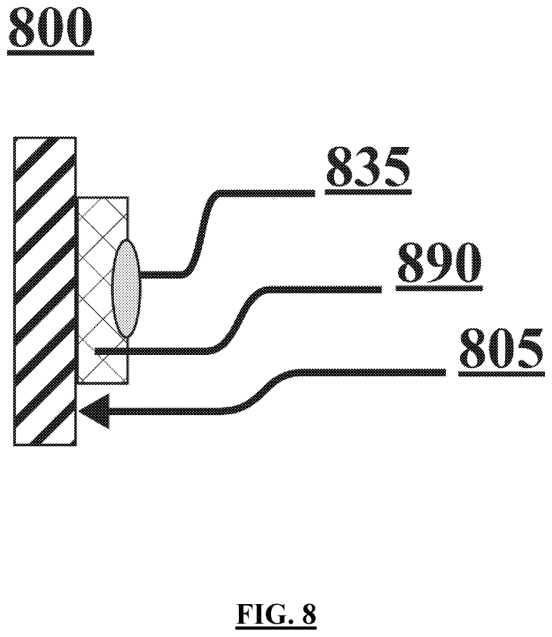

FIG. 8 shows an example use of an electrode backing 890. The bladder monitor assembly 800 comprises electrodes such as an analog circuit 120, a temperature sensor 111, a wetness sensor 145, a bioimpedance sensor(s) 135 and a drive electrode 735. Each electrode 835 is put in contact with the user's skin surface and/or held in place using force from the electrode backing 890. The electrode backing 890 may be a padding made of thick cloth, silicone, foam, or another soft material, an elastic band, a spring, or a tightened belt. The electrode backing 890 may provide force through elasticity such as a padding, an elastic band, or a spring, friction such as a rough cloth, suction by a concave surface or the like, an attachment such as a clip, snap, hinge, or buckle, or any combination thereof. In some embodiments the mounting band 805 doubles as or may be inside of the backing 890. The silicone may be in between two layers of cloth in many preferred embodiments to avoid adhesion to skin or electrical interference with the electrode by the silicone. In some embodiments, the multiple backings may be individual components. In other embodiments, multiple backings may be combined into a larger pad to hold multiple electrodes, reducing the number of components. In some cases a larger pad may be a long strip of silicone between two layers of cloth.

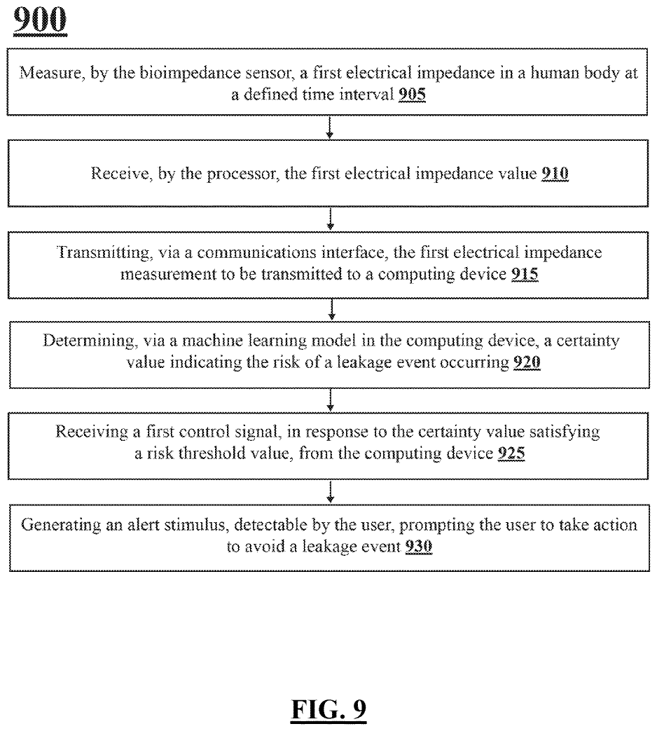

FIG. 9 illustrates a flowchart of an example method 900 for monitoring bladder conditions at a bladder monitor according to some embodiments. The method may be performed by a bladder monitoring system (such as the bladder monitor system 100 shown in FIG. 1, the bladder monitor 500 shown in FIG. 5, the bladder monitor 600 shown in FIG. 6, or the bladder monitor 700 shown in FIG. 7). The method 900 may be performed by processing logic that may include hardware (e.g., processing device, circuitry, dedicated logic, programmable logic, microcode, hardware of a device, integrated circuit, etc.), software (e.g., instructions run or executed on a processing device), or a combination thereof. In an illustrative example, the method 900 is performed by the bladder monitoring system 500 of FIG. 5 and the processing logic can be most associated with the processor 515.

Although shown in a particular sequence or order, unless otherwise specified, the order of the operations may be modified. Thus, the illustrated implementations should be understood only as examples, and the illustrated operations may be performed in a different order, while some operations may be performed in parallel. Additionally, one or more operations may be omitted in some implementations. Thus, not all illustrated operations are required in every implementation, and other process flows are possible.

At operation 905, the processing logic measures, using the bioimpedance sensor 535, a first electrical impedance in a human body at a defined timer interval.

At operation 910, the processing logic receives the first electrical impedance value.

At operation 915, the processing logic causes the first electrical impedance measurement to be transmitted to a computing device. The computing device could be either the cloud based server, 525, or the mobile device 575. The first electrical impedance value causes the computing device to calculate, using a trained machine learning model, a certainty value based on the first electrical impedance value.

In some embodiments, the machine learning model may be trained prior to the bladder monitor 510 transmitting the first electrical impedance measurement. The bioimpedance sensor 535 can measure one or more electrical impedance values in the human body and the wetness sensor 545 can measure one or more moisture levels in the human body. The bioimpedance sensor 535 and the wetness sensor 545 can transmit the measurements to the processor 515. In some embodiments, the processing logic causes the bladder monitor 510 to transmit, via the communication interface 530, the measurements to the cloud-based server 525 to be fed into the machine learning model 560. In some embodiments, the cloud-based server 525 stores the measurements as machine learning training data 565.

At operation 920, the processing logic receives a first control signal from a computing device (either the cloud based server 525, or the mobile device 575) based at least in part on the certainty value exceeding the threshold value. In embodiments, the machine learning model calculates the certainty value by taking the first electrical impedance measurement as input. The processor may compare the output of the machine learning model to the threshold value. If the certainty value exceeds the threshold value, the computing device may generate a control signal to transmit to the bladder monitor assembly 501 to alert the user of a leakage event. In some embodiments the threshold value may be determined by the user after their own trial and error or intuition. In other embodiments the threshold value may be determined by the user who consults with their healthcare provider.

At operation 925, the processing logic provides an alert stimulus to the user. In some embodiments the processing logic may transmit a first command signal to the analog circuit 520 in response to receiving the first control signal. In other embodiments the command signal is transmitted to the mobile device 575.

At operation 930, the processing logic (e.g., the analog circuit 520) generates alert stimulus in response to receiving the first command signal from processor 515. For example, the analog circuit 520 may generate a vibration or a shock upon receiving the first control signal. In other embodiments, the processing logic may cause the mobile device to provide an alert stimulus in the form of a vibration, an audible notification, or an audible alarm. The first control signal may turn on the analog circuit 520. This alert stimulus prompts the user to take action (void their urine) in order to avoid a leakage event. In other embodiments, the alert stimulus prompts the user's caregiver, such as a spouse, parent, or nanny, to take action such as waking up the user.

In some embodiments, the cloud-based server 525 is in communication with the mobile device 575. Accordingly, the cloud-based server 525 is configured to transmit to the mobile device 575, a control signal to cause the mobile device 575 to generate a notification. For example, when the cloud-based server 525 determines a leakage event is likely to occur, the cloud-based server 525 may send the control signal to the mobile device 575 to alert the user, e.g., which may be viewable or can be heard through an input/output interface such as a screen and speaker, respectively. In some embodiments, the user adjusts settings of the bladder monitor (e.g., bladder monitor assembly 501) from a phone such as the mobile device 575. For example, the user can set the method of alert (e.g., vibration, electric pulse, phone alarm, etc.), how soon before a leakage event occurs to be notified, and a power mode. The cloud-based server 525 may receive from the mobile device 575, one or more data signals including a request to reconfigure the bladder monitor 510. The cloud-based server 525 may generate, based on the one or more data signals, a second control signal. The cloud-based server 525 may transmit to the bladder monitor 510, the second control signal to cause the bladder monitor 510 to be reconfigured. For example, the one or more data signals could include a request to operate the bladder monitor 510 in a reduced power mode. Additionally, or alternatively, the one or more data signals could include a request for the alert stimulus to be a vibration or an electric pulse.

Some portions of the preceding detailed descriptions have been presented in terms of algorithms and symbolic representations of operations on data bits within a computer memory. These algorithmic descriptions and representations are the ways used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, combined, compared, and otherwise manipulated. It has proven convenient at times, principally for reasons of common usage, to refer to these signals as bits, values, elements, symbols, characters, terms, numbers, or the like.

In some embodiments a leakage event automatically causes the information from the wetness sensor 545 and the bioimpedance sensor(s) 535 to be added to the machine learning training data 565 and causes the machine learning model 560 to be retrained.

It should be borne in mind, however, that all of these and similar terms are to be associated with the appropriate physical quantities and are merely convenient labels applied to these quantities. The present disclosure can refer to the action and processes of a computer system, or similar electronic computing device, that manipulates and transforms data represented as physical (electronic) quantities within the computer system's registers and memories into other data similarly represented as physical quantities within the computer system memories or registers or other such information storage systems.

The present disclosure also relates to an apparatus for performing the operations herein. This apparatus can be specially constructed for the intended purposes, or it can include a general-purpose computer selectively activated or reconfigured by a computer program stored in the computer. Such a computer program can be stored in a computer readable storage medium, such as, but not limited to, any type of disk including floppy disks, optical disks, CD-ROMs, and magnetic-optical disks, read-only memories (ROMs), random access memories (RAMs), EPROMS, EEPROMs, magnetic or optical cards, or any type of media suitable for storing electronic instructions, each coupled to a computer system bus.

The algorithms and displays presented herein are not inherently related to any computer or other apparatus. Various general-purpose systems can be used with programs in accordance with the teachings herein, or it can prove convenient to construct a more specialized apparatus to perform the method. The structure for a variety of these systems will appear as set forth in the description below. In addition, the present disclosure is not described with reference to any particular programming language. It will be appreciated that a variety of programming languages can be used to implement the teachings of the disclosure as described herein.

The present disclosure can be provided as a computer program product, or software, that can include a non-transitory computer-readable medium having stored thereon instructions, which can be used to program a computer system (or other electronic devices) to perform a process according to the present disclosure. A computer-readable medium includes any mechanism for storing information in a form readable by a machine (e.g., a computer). In some implementations, a computer-readable (e.g., computer-readable) medium includes a machine (e.g., a computer) readable storage medium such as a read only memory (“ROM”), random access memory (“RAM”), magnetic disk storage media, optical storage media, flash memory components, etc.

In the foregoing specification, implementations of the disclosure have been described with reference to specific example implementations thereof. It will be evident that various modifications can be made thereto without departing from the broader spirit and scope of implementations of the disclosure as set forth in the following claims. The specifications and drawings are, accordingly, to be regarded in an illustrative sense rather than a restrictive sense.

What is claimed is:

1. A bladder monitor assembly comprising:
a mounting band configured to be attached to a body of a user;
a processor attached to the mounting band;
a first electrode backing attached to the mounting band;
a second electrode backing attached to the mounting band;
at least one bioimpedance sensor coupled to the processor, wherein the at least one bioimpedance sensor is attached to the first electrode backing and configured to be held in contact with a skin surface and at a first location of on the body by force from the first electrode backing, wherein the at least one bioimpedance sensor is to measure an electrical impedance value in response to a current emitted by a drive electrode held in contact with the skin surface at a second location of by the second electrode backing;
a communication interface coupled to the processor and configured to:
transmit, to a computing system, data comprising the electrical impedance value useable by the computing system as an input to a machine learning model that generates a certainty value of a leakage event; and
receive, from the computing system, a control signal in response to the certainty value satisfying a risk threshold value; and
wherein the processor is configured to cause, in response to the control signal, an alert stimulus to be generated that prompts the user to take action to avoid a leakage event.

2. The bladder monitor assembly of claim 1, further comprising a wetness sensor coupled to the processor and attached to the mounting band, the wetness sensor configured to continuously measure a moisture level, wherein the transmitted data further comprises a moisture value measured by the wetness sensor and useable, by the computing system, to train the machine learning model.

3. The bladder monitor assembly of claim 1, further comprising a wearable garment in which the mounting band is integrated.

4. The bladder monitor assembly of claim 1, wherein the first electrode backing and the second electrode backing are integrated into a silicone pad surrounded by two layers of fabric.

5. The bladder monitor assembly of claim 1, wherein the communication interface transmits the data to one of a mobile device or a cloud-based server.

6. The bladder monitor assembly of claim 1, further comprising at least one analog circuit coupled to the processor, wherein the at least one analog circuit is attached to the mounting band and configured to be held in contact with the skin surface at a third location by a third electrode backing, and wherein the at least one analog circuit is to generate an alert stimulus, detectable by the user, in response to the control signal.

7. The bladder monitor assembly of claim 1, further comprising a motion detector attached to the mounting band, wherein the motion detector measures motion values of the body, and wherein the data further comprises the motion values.

8. The bladder monitor assembly of claim 1, further comprising a temperature sensor configured to be in contact with the skin surface at a third location by a third electrode backing, wherein the temperature sensor measures the temperature of the user, and wherein the data further comprises the temperature of the user.

9. A method of monitoring bladder conditions, the method comprising:
measuring, by a bioimpedance sensor attached to an electrode backing which keeps the bioimpedance sensor in contact with a surface of a human body of a user, one or more electrical impedance values from the human body;
receiving, by a processor coupled to a bladder monitor assembly, the one or more electrical impedance values;
transmitting, via a communication interface, to a computing system, the one or more electrical impedance values;
cleaning, at the computing system, the one or more electrical impedance values based on one or more motion values from a motion detector;
causing the one or more electrical impedance values to be fed into a machine learning model, executed on the computing system, that determines a certainty value indicating a risk of a leakage event occurring;
receiving, from the computing system, a first control signal in response to the certainty value satisfying a first risk threshold value; and
in response to the first control signal, generating an alert stimulus, by the bladder monitor assembly and that is detectable by the user, prompting the user to take action to avoid a leakage event.

10. The method of claim 9, the method further comprising:
measuring, by a wetness sensor of a bladder monitor assembly attached to a mounting band, one or more moisture values; and
transmitting, via the communication interface, the one or more moisture values to also be fed into the machine learning model when determining the certainty value of a leakage event occurring.

11. The method of claim 9, wherein the computing system is one of a cloud-based server or a mobile device that is communicatively coupled with the bladder monitor assembly.

12. The method of claim 9, wherein generating the alert stimulus comprises transmitting an alert signal to a mobile device of the user that triggers the mobile device to generate an alert stimulus, detectable by the user, prompting the user to take the action to avoid the leakage event.

13. The method of claim 9, wherein the alert stimulus is one of a vibration generated by the bladder monitor assembly or an electric pulse generated by an analog circuit attached to a mounting band.

14. The method of claim 9, further comprising:
generating, on a mobile device, one or more data signals comprising a request to reconfigure a bladder monitor of the bladder monitor assembly;
generating, based on the one or more data signals, a second control signal; and
transmitting, to the bladder monitor, the second control signal to cause the bladder monitor to be reconfigured according to the one or more data signals.

15. The method of claim 9, wherein the certainty value is determined without calculating a bladder volume.

16. The method of claim 9, further comprising:
receiving, via a mobile device of the user, a second risk threshold value; and
replacing the first risk threshold value with the second risk threshold value as a condition for generating the first control signal.

17. A computing system comprising:
one or more processing devices; and
non-transitory computer-readable storage medium storing instructions, which when executed by the one or more processing devices, causes the one or more processing devices to perform operations comprising:
receiving measurement data comprising one or more electrical impedance values from a bladder monitor assembly comprising at least one bioimpedance sensor, which measures electrical impedance values, held in contact with a skin surface of a user by an electrode backing;
inputting the measurement data into a machine learning model;
executing the machine learning model to determine a certainty value of a leakage event occurring; and
generating and transmitting, to the bladder monitor assembly, a control signal in response to the certainty value satisfying a risk threshold value, the control signal configured to trigger, one of the bladder monitor assembly or a mobile device, to generate an alert stimulus detectable by the user.

18. The computing system of claim 17, wherein the measurement data further comprise one or more moisture values indicating a leakage event occurred in response to the one or more moisture values exceeding a moisture value threshold, and wherein the operations further comprises training the machine learning model based on the one or more moisture values.

19. The computing system of claim 17, wherein the measurement data further comprises an input indicator, received from a mobile device of the user, indicating when a leakage event occurred, and wherein the operations further comprise integrating the input indicator into supervised training of the machine learning model.

20. The computing system of claim 17, wherein the operations further comprise retraining the machine learning model automatically upon one of the one or more processing devices detecting a leakage event or after passage of a predetermined amount of time.
